(19) United States
(12) Patent Application Publication
(10) Pub. No.: US 2011/0213275 A1
(43) Pub. Date: Sep. 1, 2011

(54) DEVICE FOR DETERMINING THE STABILITY OF A KNEE JOINT

(75) Inventors: Ronald Boos, Kaisheim (DE); Gerhard Marquardt, Tubingen (DE); Urs Schneider, Stuttgart (DE); Wolfgang Bort, Waiblingen (DE)

(73) Assignee: BORT GMBH, Wienstadt-Benzach (DE)

(21) Appl. No.: 13/001,065

(22) PCT Filed: Jun. 18, 2009
(86) PCT No.: PCT/EP2009/004417

§ 371 Date: May 9, 2011

(30) Foreign Application Priority Data
Jun. 27, 2008 (DE) 10 2008 030 534.0

(51) Int. Cl. A61B 5/11

(52) U.S. Cl. 600/595

(57) ABSTRACT

The invention relates to a device for determining the stability of a knee joint. The device comprises includes a measuring sensor (14), which can be attached via a fastening device (16) to a lower leg (12) associated with the knee joint (11). The measuring sensor (14) is designed to measure an acceleration in at least one direction (z) during a movement of the lower leg (12). Furthermore, a processing device (18) is provided for processing measured values of the measuring sensor (14) in order to infer the stability of the knee joint (11) from the processed measured values.

### BACKGROUND

1. Technical Field

The invention relates to a device for determining the stability of a knee joint with at least one contactless measuring sensor.

2. Description of Related Art

The cruciate ligaments of the human knee joint stabilise the femur (thigh bone) with respect to the tibia (shin bone). Like all ligaments in the human body, however, they can only take tensile forces. To stabilise the knee joint, therefore, there are two cruciate ligaments which run in opposite directions to one another.

If in the event of a cruciate ligament rupture (cruciate ligament tear), the anterior cruciate ligament, for example, is severed, it can no longer take tensile forces. Since the posterior is cruciate ligament cannot take compressive forces, the knee joint becomes unstable. Consequently, after such a cruciate ligament rupture, it is possible for the shin bone to move, through tension, further in the “forward” direction with regard to the thigh than is possible in a healthy knee joint.

Methods for testing the stability of a knee joint are carried out above all for pre- and postoperative diagnostics in the course of therapy of cruciate ligament ruptures.

In the so-called “Lachmann test”, with a patient lying the knee joint to be diagnosed is held at a flexion angle of the knee joint of approximately 30°. Subsequently, the therapist grasps the lower leg with both hands in such a way that both index fingers lie in the hollow of the knee. The therapist then pulls the lower leg forwards. A partial rupture or complete rupture of the cruciate ligament can be diagnosed depending on the displaceability of the lower leg with respect to the thigh.

A similar diagnostic method is used in the so-called “anterior drawer test”. The anterior drawer test differs from the Lachmann test mainly in that the knee joint to be diagnosed is examined at a flexion angle of approximately 90°. The displacement distance between the thigh and lower leg is referred to as the “drawer travel”.

To determine the rotational stability of the knee joint, use is made of the so-called “pivot-shift test”, also known as the subluxation test. This diagnostic method is employed, inter alia, to examine the knee joint when there is a suspected tear or injury of the anterior cruciate ligament. The pivot-shift test is also employed for therapy following a novel “double-bundle operation” owing to a cruciate ligament rupture, where the rotational stability of the knee joint is restored.

In the pivot-shift test, the therapist pushes the lower leg of the lying patient, with the knee bent, downwards with one hand and at the same time internally rotates the lower leg. The test is considered positive if rearward sliding of the upper tibial plateau (shin-bone head) occurs. In particular, at a flexion angle of the knee joint of between 27° and 45°, the therapist perceives a sudden snapping, when the lateral femoral condyle springs forwards with respect to the lateral tibial condyle. This phenomenon is also visible externally in some cases.

However, Lachmann, anterior drawer and pivot-shift tests carried out by a therapist have the disadvantage that the results of the examination depend purely subjectively on the assessment and experience of the therapist.

For this reason, a series of device-assisted diagnostic methods have been developed with the intention of improving the accuracy of the subjective, manual diagnostic methods.

In the radiological Lachmann test, the above-described Lachmann test is verified using a knee-holding device and an X-ray apparatus. However, this method has the disadvantage that the patient is exposed to considerable radiation.

In addition, magnetic resonance tomography is also used to verify the diagnostic methods. However, for routine diagnostics in small surgical outpatient departments or practices, magnetic resonance tomographs are generally too expensive and require considerable installation space and technical resources.

Furthermore, devices for instrumental measurement, so-called arthrometers, for determining the stability of a knee joint with the aid of the Lachmann test are known. With these devices, a manually exerted force for the ventral translation of the tibia in comparison with the patella of the knee joint is mechanically exerted. The measurement results are determined purely mechanically with the aid of the device and displayed via scales. The use of such devices is, however, awkward in some cases, since the devices have to be fastened to the patient's leg at more than one place. Moreover, the devices are of a certain size, making them difficult to handle. Also, the accuracy of the measurement results is often not sufficient. Furthermore, with known arthrometers it is only possible to carry out the Lachmann test. Determination of the rotational stability of the knee joint cannot be performed with such known devices. This is due in particular to the fact that the rotational stability of the knee joint can be produced only to a limited degree with older operating techniques.

The document U.S. Pat. No. 4,583,555 relates to a device for mechanically measuring the displaceability of a lower leg with respect to the associated knee joint in the Lachmann test.

The document U.S. Pat. No. 4,649,934 relates to a device for measuring the mobility of a knee joint comprising a treatment chair with a built-in dynamometer.

A joint diagnosis set for detecting and evaluating the movement of a knee joint with a marker system is known from the document DE 201 18 040 U1.

The document DE 39 25 014 A1 relates to a device for testing the stability of a knee joint with a holding device having two partial plates connected to one another in articulated fashion and a computer-aided ultrasonic apparatus which is pressed into the soft parts of a clamped knee joint.

The document DE 197 01 838 A1 relates to a device for determining the stability of a knee joint with two distance sensors designed as linear potentiometers.

The document DE 36 36 843 A1 relates to a device for determining the stability of a knee joint comprising a chair with a greatly indented seat for fixing the pelvis.

Against this background, an object of the present invention is to provide a device for determining the stability of a knee joint which enables the translational stability in a sagittal plane and/or the rotational stability about an axis in the horizontal plane of the knee joint to be determined in a manner which is simple and uncomplicated to handle.

### SUMMARY

This object is achieved by a device for determining the stability of a knee joint, having a measuring sensor, which can be attached via a fastening device to a lower leg associated with the knee joint, the measuring sensor being designed to measure an acceleration in at least one direction during a movement of the lower leg, and a processing device being provided for processing measured values of the measuring sensor in order to infer the stability of the knee joint from the processed measured values.

In the case of the device for determining the stability of a knee joint according to the present invention, it is possible to make a statement regarding the stability of the knee joint with the aid of the merely one measuring sensor, which is attached via a fastening device to a lower leg associated with the knee joint, and of the processing device for processing the measured values of the measuring sensor. The handling of the device is substantially simplified by this simple structure with only a few components. In particular, the therapist does not have to operate any element of the device while carrying out a diagnostic method and thus has both hands free for the diagnostic method.

The processing device for processing measured values of the measuring sensor can be a computer with a computer program for evaluating the measurement results, a storage device for storing the measurement results and a display for displaying the measurement results and evaluating the measurement results.

The measuring sensor for measuring an acceleration and/or angular velocity in at least one direction during a movement of the lower leg is preferably an inertial sensor. An inertial sensor determines the changes of orientation and position based on an inertial navigation system (INS). In this system, the sensor's own orientation, position and velocity are determined without the need for reference to the external surroundings.

The inertial sensor can comprise acceleration sensors and rotation rate sensors, so-called gyroscopes, for all three spatial directions. The rotation rate sensors determine angular velocities about an axis of rotation during a movement. The inertial sensor can determine the accelerations in three spatial directions and the angular velocities about three spatial axes. A change of position of the inertial sensor can be calculated from the acceleration and angular-velocity values determined. Inertial sensors have the advantage that they are robust, manage without an infrastructure or reference values and are insensitive to shadowing and interference.

The at least one direction during a movement of the lower leg is preferably a direction of movement of the lower leg during flexion and extension of the knee joint and a direction substantially perpendicular to the direction in which the lower leg extends. In particular, the direction can be a direction of movement of the lower leg in the Lachmann test.

A distance of the movement travel of the lower leg can be determined by calculation with the aid of double integration of the acceleration value from the acceleration value determined with the aid of the measuring sensor in at least one direction. As a result, in the Lachmann test for example, based on an acceleration measurement, a statement can be made about the degree of displaceability of the lower leg in relation to the thigh, i.e. the stability of the knee joint. The movement travel can be, in particular, the drawer travel defined at the outset. The integration of the acceleration values can be performed from a certain acceleration value. This may be advantageous in order to exclude from the evaluation acceleration which is not athibutable to the diagnostic method.

The processing device can determine a flexion angle of the knee joint with the aid of a value of the acceleration and a reference value. The reference value can be, for example, a flexion angle of 180° with the leg stretched out. Furthermore, for the calculation of the flexion angle, it can be assumed that the patient is lying on a horizontal plane, for example a couch, with the knee flexed. The calculation can, moreover, be based on the lower leg and the thigh having the same length. The flexion angle of the knee joint can thus be calculated via the equilateral triangle formed between the thigh, lower leg and supporting surface.

Such a calculation of the flexion angle of the knee joint eliminates the need for a separate determination of the flexion angle with a manual angle-measuring device while carrying out a diagnostic method. For example, the Lachmann or pivot-shift test has to be carried out at specific flexion angles of the knee joint. By displaying the calculated flexion angle on a display device (for example a screen provided in the processing device) while carrying out the diagnostic method, the therapist can monitor constantly, i.e. during the diagnosis, and without additional actions, whether the knee joint is flexed at the desired angle or can appropriately adjust the flexion angle of the knee joint. The flexion angle which has been calculated or stored in the processing device can also be used during a subsequent evaluation of the measurement results in order to distinguish between different diagnostic methods carried out on the knee joint, for example a transition from the Lachmann to the pivot-shift test.

The processing device can furthermore be configured to determine extremes of a plurality of acceleration values during the movement of the lower leg. An instability of the knee joint can be inferred from the extremes of the acceleration values, in particular in the pivot-shift test. Experiments have shown that characteristics of an unstable knee joint can be identified in particular from the acceleration peak values, especially on comparison with a healthy knee joint.

The processing device can also be configured to determine a rotation angle of the lower leg during a rotational movement of the lower leg. For this purpose, the angular velocity during a rotation of the lower leg can be determined by the measuring sensor with the aid of a rotation rate sensor or a gyroscope and the rotation angle can be calculated by single integration from the angular velocity. The determination of the rotation angle of the lower leg can be used in particular in the pivot-shift test to ascertain whether the lower leg has been rotated as far as a desired rotation angle. It is also conceivable to evaluate or calculate and display the angular velocity and/or the angular acceleration during a rotational movement of the lower leg in the processing device, in order to be able to draw conclusions about the rotational stability of the knee joint.

The device for determining the stability of a knee joint can furthermore have a second measuring sensor, which can be attached via a second fastening device to a thigh associated with the knee joint, the second measuring sensor being designed to measure an acceleration in at least one direction during a movement of the thigh.

The second measuring sensor can likewise be an inertial sensor. With the aid of the measurement results of the first and second measuring sensor, an even more accurate determination of the stability of the knee joint is possible, for example in the Lachmann or pivot-shift test, owing to the additional acceleration and angular-velocity values. As in the case of the measuring sensor provided for the lower leg, the second measuring sensor can determine accelerations in each case in three spatial directions and angular velocities about three spatial axes. These measured values can be used by the processing device for the determination of the stability of the knee joint. With the aid of the additional measured values of the second measuring sensor, it is also possible to carry out an even more accurate determination of the flexion angle of the knee joint than with only one measuring sensor, where the calculation of the flexion angle takes into account a reference value.

The object of the present invention stated at the outset is also achieved by a device for determining the stability of a knee joint having a contactless measuring sensor, which can be attached via a fastening device to a lower leg associated with the knee joint, the contactless measuring sensor being designed to measure a distance between the contactless measuring sensor and a reference point during a movement of the lower leg, and a processing device being provided for processing measured values of the measuring sensor in order to infer the stability of the knee joint from the processed measured values.

The use of a contactless measuring sensor for measuring a distance has the advantage that a device for determining the stability of a knee joint can be provided with a simple structure, thus simplifying the handling of the device.

The contactless measuring sensor can comprise a laser sensor, an ultrasonic sensor and/or an infrared sensor. Such contactless measuring sensors have the advantage that only the sensors and no essential additional components are necessary for the distance measurement. As a result, the complexity of the device is reduced.

The reference point can be located on the knee joint to be examined. The reference point is preferably located centrally on the kneecap of the knee joint. The reference point can, however, also be located on the thigh associated with the knee joint or on the treatment couch.

As a result of the fact that the position of the lower leg or the shin bone with the contactless measuring sensor firmly attached thereto changes relative to the motionless knee joint, i.e. the kneecap, in the Lachmann or pivot-shift test for example, the distance between the contactless measuring sensor and the reference point also changes. Consequently, the distance between measuring sensor and reference point can be determined in a simple and precise manner with the aid of the contactless measuring sensor.

The fastening device is preferably designed in such a way that it maintains the contactless measuring sensor at a short distance above the knee joint, i.e. in particular the kneecap, or the thigh associated with the knee joint. As a result, the distance between the measuring sensor and the reference point during a movement of the thigh, in the Lachmann or pivot-shift test for example, can be determined.

Further preferably, the fastening device extends substantially parallel to the direction in which the lower leg extends. This embodiment is advantageous since in most diagnostic methods, such as the Lachmann or pivot-shift test for example, the lower leg is moved and the fastening device thus follows in parallel the movement of the lower leg.

According to one embodiment, the fastening device comprises at least one holding shell with fastening means for fastening the holding shell to the lower leg. For an accurate determination of the acceleration or the distance between the contactless measuring sensor and the reference point, it is crucially important for the measuring sensor to be moved together with the lower leg. For this reason, it is necessary for the measuring sensor to be firmly connected to the lower leg and to be unable to slip, tilt or otherwise move in relation to the lower leg. The holding shell ensures a high tilting and rotational stability. Furthermore, the holding shell can be matched to the anatomy of the human lower leg, it being possible for the holding shell to be fastened to the lower leg with the aid of fastening means. The fastening means can be a crepe fastener. The fastening means can also comprise a hook attached to a flexible strip, which can be hung onto an eye element on the medial side of the tibia. The holding shell can be designed in such a way that it is attachable both to the left and right lower leg.

According to a development of the invention, the fastening device comprises two holding shells which can be fastened to the lower leg with the aid of fastening means. Since the anatomy of different lower legs differs, the fastening means is preferably designed in such a way that the holding shell can be firmly attached to the lower leg irrespective of the shape thereof.

In order to enable a further-simplified handling of the device for determining the stability of a knee joint, the measured values of the measuring sensors are transmitted wirelessly, in a wired manner or via a storage medium to the processing device. Since the therapist has to grasp or move the lower leg or the thigh when carrying out the diagnostic methods, a wired connection between the measuring sensor and the processing device would make it more difficult to carry out the diagnostic methods.

Preferably, the measuring sensor measures a plurality of measured values over time. The plurality of measured values over time are transmitted to the processing device and evaluated by the latter. By determination of the measured values over time, a statement regarding the stability of the knee joint can be made, in particular in the Lachmann or pivot-shift test. Further advantageously, the measured values over time can be used for a comparison of the knee to be diagnosed with a healthy knee.

According to a further embodiment of the invention, the processing device averages the measured values over a predetermined time interval. By the averaging, measurement errors can be suppressed. Other smoothing methods may also be employed.

Preferably, the movement of the lower leg is a translational and/or rotational movement of the lower leg. The device is furthermore configured to determine the translational stability in a sagittal plane and/or the rotational stability about an axis in the horizontal plane of the knee joint.

The device according to the present invention is thus capable of determining both a translational stability of the knee joint, in the Lachmann test for example, and a rotational stability of the knee joint, in the pivot-shift test for example. Consequently, according to the present invention, only one device is needed to carry out more than one diagnostic method on a knee joint.

The object stated at the outset is also achieved by a system for determining the stability of a knee joint having a device for determining the stability of a knee joint with a measuring sensor for measuring an acceleration in at least one direction during a movement of the lower leg and a device for determining the stability of a knee joint with a contactless measuring sensor, for example a laser sensor. Even more accurate measurement results can be obtained by such a combination. Furthermore, an even more accurate statement regarding the stability of the knee joint can be made. Moreover, one of the devices can be used as a reference measuring device for the other device. One of the devices can also be used for calibrating the other devices. Furthermore, the measurement results of both devices can also be compared in order to be able to make a statement regarding the accuracy of one device.

### BRIEF DESCRIPTION OF THE DRAWINGS

The invention is explained by way of example below with reference to the accompanying figures, in which:

FIG. 1 shows a schematic illustration of a device according to the invention for determining the stability of a knee joint with an inertial sensor;

FIG. 2 shows a schematic illustration of the measuring directions on a thigh and a lower leg;

FIG. 3 shows a schematic illustration of a device according to the invention for determining the stability of a knee joint with two inertial sensors; and

FIG. 4 shows a schematic illustration of a device according to the invention for determining the stability of a knee joint with a laser sensor.

### DETAILED DESCRIPTION

Exemplary embodiments of the present invention when carrying out the Lachmann or pivot-shift test are explained below. However, the present invention is not restricted to a use in the Lachmann or pivot-shift test. In principle, the present invention can be used in any diagnostic method in which the lower leg and/or the thigh is moved.

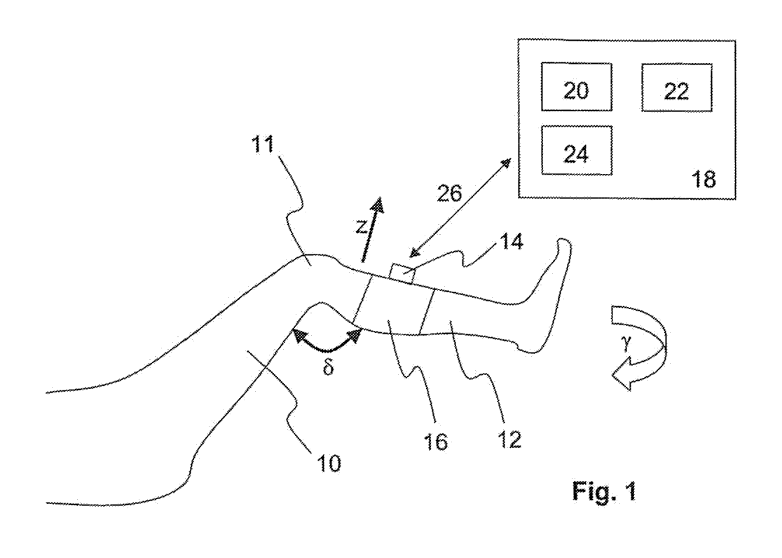

FIG. 1 shows a schematic illustration of a human leg with a device according to the invention for determining the stability of a knee joint. Both the translational stability and the rotational stability of a knee joint can be determined with the device shown.

The leg comprises a thigh 10, a knee joint 11 to be diagnosed and a lower leg 12.

An inertial sensor 14 is fastened to the lower leg 12 with the aid of a fastening device 16. Furthermore, a processing device 18 for processing the measured values of the inertial sensor 14 is provided. The processing device 18 comprises a display 20, a storage device 22 and processing logic 24.

In the Lachmann test, the lower leg 12 is moved relative to the thigh 10 in the direction z. The acceleration of the movement of the lower leg 12 in the direction z is measured by the inertial sensor 14 and the measured values are transmitted to the processing device 18 in a wired or wireless manner. The movement travel of the lower leg 12 relative to the thigh 10 is the drawer travel.

In the pivot-shift test, the lower leg 12 is rotated in the direction of rotation γ and flexed. The inertial sensor 14 determines the angular velocity of the rotation. The measured values are transmitted from the inertial sensor 14 to the processing device 18. Further acceleration and angular-velocity values can be measured by the inertial sensor 14 and transmitted to the processing device 18. In order not to hinder the carrying-out of the diagnostic methods, the measured values are transmitted wirelessly 26 from the inertial sensor 14 to the processing device 18.

In the processing device 18, the received measured values are stored in the storage device 22 and processed by processing logic 24. With the aid of a double integration of the measured acceleration values, the processing logic 24 determines the displacement travel of the lower leg 12 during a movement thereof relative to the thigh 10 or the knee joint 11. On the basis s of the displacement travel in the Lachmann test, it is possible to conclude whether a translational instability of the knee joint is present.

The movement of the lower leg 12 may be both a translational and a rotational movement of the lower leg 12. A rotational instability of the knee joint 11 can thus be inferred from the acceleration values during a rotation of the lower leg 12, in particular from the peak values of the acceleration. In particular, the sudden snapping in the pivot-shift test, when the lateral femoral condyle springs forwards with respect to the lateral tibial condyle, is evident from a sudden change of the acceleration or acceleration peak values and sudden rotations.

The processing logic 24 can also determine the angle of rotation during a rotation of the lower leg 12 by single integration of the angular-velocity value. The rotation angle can be displayed in the display 22 while carrying out the diagnostic method, so that this angle assists the therapist with regard to the rotation of the lower leg 12 while carrying out the pivot-shift test.

With the aid of the measured acceleration and angular-velocity value during a translational movement of the lower leg 12 in the direction z, the processing logic 24 can also calculate the flexion angle δ of the knee joint 11 between the thigh 11 and the lower leg 12. The calculation can in this case take into account a starting angle δ of 180° on extension of the knee joint 11 and the substantially equal length of the thigh 10 and lower leg 12. In particular, the calculation can be performed with the aid of the assumption of an equilateral triangle, where the thigh 10 and the lower leg 12 form the equal sides of the triangle.

All the measured and calculated values are stored in the storage device 22 together with a time mark. All the values can also be displayed in the display 20.

By displaying the calculated flexion angle δ while carrying out a diagnostic method, for example the Lachmann test, the therapist can see while carrying out the diagnostic method whether the knee joint 11 is flexed at the flexion angle recommended for the particular test.

In the analysis of whether an instability of the knee joint (i.e. a ligament tear) is present, or to what degree the knee joint is unstable, the measured values displayed in the display 20 can be analysed over time. It can thus also be determined from maximum acceleration values whether the rotational stability of the knee joint 11 is impaired. This determination can also be performed automatically by the processing logic 24. For simplified presentation of the results of the analysis or to reduce measurement errors, the processing logic 24 can furthermore carry out an averaging of the measured values.

To ascertain whether the translational stability and/or the rotational stability of the knee joint is impaired, the measurements can be carried out both on the knee joint to be diagnosed and on the other (healthy) knee joint. The measured values of both knee joints are stored in the storage device 22, processed by the processing logic 24 and subsequently displayed in the display 20 for comparison. This comparative presentation or juxtaposition of the measurement results of the knee joint to be diagnosed and the other knee joint enables the therapist to ascertain in a simple manner whether the knee joint to be diagnosed is unstable.

The processing logic 24 can also be designed in such a way that it analyses the measured values automatically and informs the therapist via the display 20 whether an instability of the knee joint is present or to what degree the knee joint is unstable.

The inertial sensor 14 is firmly connected to the fastening device 16. In this regard, the inertial sensor 14 can be attached to the fastening device 16 directly or via a connecting element (not shown). The fastening device 16 comprises one or two shell elements (not shown), which can be firmly attached to the lower leg with the aid of a crepe or touch-and-close fastener (not shown) capable of being tightened. The firm connection of the fastening device 14 to the lower leg 12 ensures that the inertial sensor 14 does not deliver incorrect measurement results due to self-movements. The fastening device 16 may also comprise a touch-and-close strip, to which the inertial sensor 14 is fastened.

In the embodiment according to FIG. 1, only a measurement of the acceleration of the lower leg 12 in the direction z and of the angular velocity in the direction γ during a rotation of the lower leg 12 has been described in detail. However, the present invention is not restricted to these measured quantities, measuring directions and movement directions of the lower leg 12.

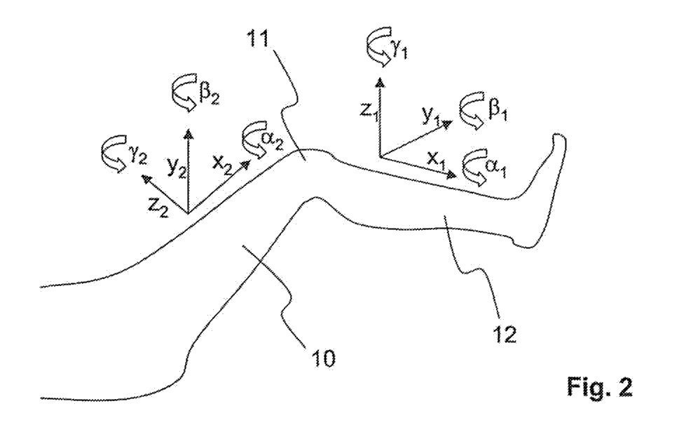

FIG. 2 thus shows a schematic illustration of the measured quantities and measuring directions which can be determined on a thigh and a lower leg. In particular, FIG. 2 shows the coordinates of an inertial navigation system, in each case on the lower leg 12 and the thigh 10.

The accelerations in the directions x₁, y₁ and z₁ can thus be determined with an inertial sensor (not shown in FIG. 2) attached to the lower leg 12. Furthermore, the inertial sensor can determine the angular velocities in the directions of rotation α₁, β₁ and γ₁. For this purpose, the inertial sensor can comprise three acceleration sensors and three gyroscopes.

The same measured values, i.e. accelerations in the directions x₂, y₂ and z₂ and angular velocities in the directions of rotations α₂, β₂ and γ₂ can also be determined with the aid of a further inertial sensor (not shown) attached to the thigh 10.

All the measured values determined by the inertial sensors can be transmitted to the processing device 18 shown in FIG. 1 and further processed there.

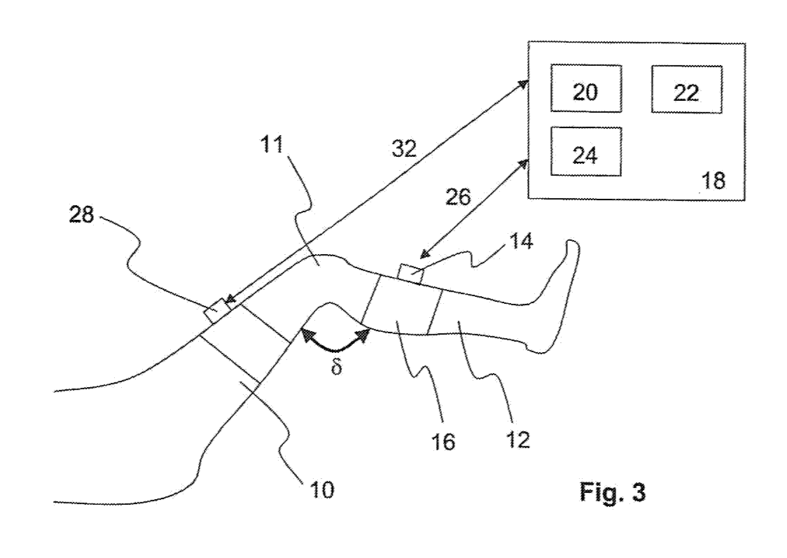

The embodiment according to FIG. 3 shows a schematic illustration of a device according to the invention for determining the stability of a knee joint with two inertial sensors. The embodiment according to FIG. 3 differs from the embodiment according to FIG. 1 in that a second inertial sensor 28 is provided. Identical elements in FIGS. 1 and 3 have the same reference symbols and these elements are not described again below.

The second inertial sensor 28 is firmly attached to the thigh 10 with the aid of a fastening device 30. The inertial sensor 28 corresponds to the inertial sensor 14 and measures acceleration values and angular velocities during a translational and/or rotational movement of the lower leg 12 which also affects the movement of the thigh 10. The inertial sensor 28 sends 32 its measured values wirelessly to the processing device 18. The measured values from the inertial sensor 14 and from the inertial sensor 28 are processed in the processing device 18. In particular, the processing logic 24 processes the measured values, the storage device 22 stores the measured values and the display 20 displays the processed measured values.

It is possible to determine the flexion angle δ of the knee joint 11 with the aid of the measured values of the second inertial sensor 28. Moreover, acceleration and velocity measured values of the inertial sensor 28 can also be used, in addition to the measured values determined by the inertial sensor 14, to make a more accurate statement regarding the stability of the knee joint 11 during a translational excursion or rotation of the lower leg 12.

The fastening device 30 for fastening the second inertial sensor 28 to the thigh 10 can be designed exactly like the fastening device 16 for fastening the first inertial sensor 14. What is important here is that the second inertial sensor 28 is firmly held on the thigh 10 in order to avoid measurement inaccuracies due to self-movements of the second inertial sensor 28 relative to the thigh 10.

The fastening device 16 is preferably fastened to the lateral and/or medial tibial head and to the medial tibial shaft. The fastening device 30_{[R1]} is preferably fastened laterally and/or medially above the condyles.

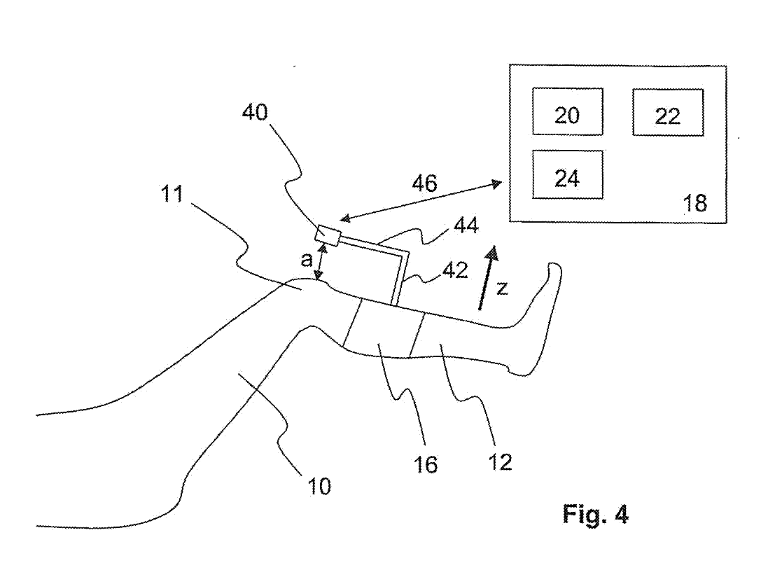

FIG. 4 shows a schematic illustration of a device according to the invention for determining the stability of a knee joint 11 with a laser sensor 40 which is attached to the lower leg 12 via a fastening device 16, 42, 44. Identical elements to FIG. 1 are again denoted by the same reference symbols and these elements are not described again below.

In this embodiment, the laser sensor 40 is provided for determining a distance a between the laser sensor 40 and a reference point. In particular, the distance a is determined during a movement of the lower leg 12. The reference point is the kneecap of the knee joint 11.

The laser sensor 40 is firmly attached to the lower leg 12 with the aid of two holding struts 42, 44 arranged substantially at right angles to one another and two holding shells 16 placed around the lower leg 12. The holding strut 42 is firmly connected to at least one of the holding shells 16 and is situated substantially perpendicularly to the direction in which the lower leg 12 extends. The holding strut 44 runs substantially parallel to the direction in which the lower leg 12 extends. The laser sensor 40 is attached to an end of the holding strut 44. The laser sensor 40 is firmly held at a short distance above the kneecap of the knee joint 11 with the aid of the fastening device 16, 42, 44. The short distance a can be initially a distance of approximately 10 cm.

If a Lachmann test is being carried out, i.e. the lower leg 12 is displaced relative to the thigh 10 in the direction z, the distance a between the laser sensor 40 and the kneecap of the joint 11 changes.

The measured values of the distance a between the laser sensor 40 and the kneecap of the knee joint 11 are transmitted wirelessly 46 from the laser sensor 40 to the processing device 18. The measured values are processed in the processing device 18 by the processing logic 24, stored in the storage device 22 and displayed by the display 20.

The measurement can also be performed during a rotation of the lower leg 12, for example in the pivot-shift test. By means of the storage and subsequent analysis of the distance a over time by the processing device 18, a rotational instability can also be detected. In particular, the sudden snapping in the pivot-shift test, when the lateral femoral condyle springs forwards with respect to the lateral tibial condyle, can be detected from a sudden change of the measured values, i.e. of the distance a.

The measurements can be carried out on the knee joint to be diagnosed and the other knee joint. Measured values stored in the storage device 22 can subsequently be compared and evaluated. In particular, the measured values can be displayed in a juxtaposed manner in the display 20.

With the aid of the device for determining the stability of a knee joint according to FIG. 4, it is possible to make a statement regarding the translational stability and rotational stability of the knee joint 11 merely based on the measurement of the distance a between a laser sensor 40 firmly attached to the lower leg 12 and a reference point, in particular the kneecap of the knee joint 11. The processing device 18 can also be designed in such a way that it analyses the measured values automatically and informs the therapist on the display 20 whether an instability of the knee joint is present or to what degree the knee joint is unstable.

The present invention is not restricted to laser sensors. For instance, in principle any kind of contactless measuring sensors, such as for example ultrasonic sensors or infrared sensors, may be used. Use of cameras, markers, potentiometers, magnetic-field sensors, strain gauges, measurement via fluid displacement, capacitive and/or inductive measurement, is also possible instead of or in addition to the laser sensor or the inertial sensor.

The devices according to the invention can also be combined in order to deliver even more accurate measurement results or to calibrate a measuring method or make statements regarding accuracy of a certain measuring device. For instance, a device can comprise both an inertial sensor and a laser sensor.

The devices according to the present invention have the advantage that accurate statements can be made regarding the stability of a knee joint, in particular in the case of a cruciate ligament tear or before or after an operation of a cruciate ligament tear.

The devices for determining the stability of a knee joint according to the present invention are small devices which are easy to handle, inexpensive and have few components and which can be used in every relatively small medical practice or outpatient department.

In particular, the devices can be employed to improve the chances of healing after a double-bundle knee-joint operation in which the rotational stability of the knee joint is restored.

With the aid of the devices according to the invention, it is possible to make a statement regarding the stability of the knee joint in both the Lachmann test and the pivot-shift test.

As a result of the wireless transmission of the measured values between the measuring sensor and the processing device, the carrying-out of the diagnostic methods is not hampered. In particular, the therapist is able to use both hands in the diagnostic methods. The results could also be displayed directly on the apparatus.

1. Device for determining the stability of a knee joint, comprising:
a measuring sensor, which can be attached via a fastening device to a lower leg associated with the knee joint, the measuring sensor being designed to measure an acceleration in at least one direction during a movement of the lower leg, and
a processing device being provided for processing measured values of the measuring sensor in order to infer the stability of the knee joint from the processed measured values, wherein the measuring sensor includes at least one inertial sensor.

2. (canceled)

3. Device according to claim 1, wherein the at least one direction is a direction of movement of the lower leg during flexion and extension of the knee joint and a direction substantially perpendicular to the direction in which the lower leg extends.

4. Device according to claim 1, wherein the processing device is configured to determine a distance of the movement travel of the lower leg with the aid of double integration of a value of the acceleration.

5. Device according to claim 1, wherein the processing device is configured to determine a flexion angle of the knee joint with the aid of a value of the acceleration and a reference value.

6. Device according to claim 1, wherein the processing device is configured to determine extremes of a plurality of acceleration values during the movement of the lower leg.

7. Device according to claim 1, wherein the processing device is configured to determine a rotation angle of the lower leg during a rotational movement of the lower leg.

8. Device according to claim 1, further comprising:
a second measuring sensor, which can be attached via a second fastening device to a thigh associated with the knee joint, the second measuring sensor being designed to measure an acceleration in at least one direction during a movement of the thigh.

9. Device according to claim 8, wherein the processing device is configured to determine a flexion angle of the knee joint with the aid of the measurement results of the two measuring sensors.

10. Device for determining the stability of a knee joint, comprising:
a contactless measuring sensor, which can be attached via a fastening device to a lower leg associated with the knee joint, the contactless measuring sensor being designed to measure a distance between the contactless measuring sensor and a reference point during a movement of the lower leg, and a processing device being provided for processing measured values of the measuring sensor in order to infer the stability of the knee joint from the processed measured values, wherein the processing device is adapted to calculate an average value of the measured values over a predetermined time interval.

11. Device according to claim 10, wherein the contactless measuring sensor includes a laser sensor, an ultrasonic sensor and/or an infrared sensor.

12. Device according to claim 10, wherein the reference point is located on the knee joint or on the thigh associated with the knee joint.

13. Device according to claim 10, wherein the fastening device maintains the contactless measuring sensor at a short distance above the knee joint or the thigh associated with the knee joint.

14. Device according to claim 10, wherein the fastening device extends substantially parallel to the direction in which the lower leg extends.

15. Device according to claim 1, wherein the fastening device includes at least one holding shell with fastening means for fastening the holding shell to the lower leg.

16. Device according to claim 1, wherein the measured values are transmitted wirelessly to the processing device.

17. Device according to claim 1, wherein the measuring sensor measures a plurality of measured values over time.

18. (canceled)

19. Device according to claim 1, wherein the movement of the lower leg is a translational and/or rotational movement of the lower leg.

20. Device according to claim 1, wherein the device is configured to determine the translational stability in a sagittal plane and/or the rotational stability about an axis in the horizontal plane of the knee joint.

21. System for determining the stability of a knee joint having a device according to claim 1.

22. System for determining the stability of a knee joint comprising a device according to claim 10.
